(19) United States
(12) Patent Application Publication
(10) Pub. No.: US 2003/0166593 A1
(43) Pub. Date: Sep. 4, 2003

(54) Non-viral vesicle vector for cardiac specific gene delivery

(76) Inventors: Kenneth Chien, La Jolla, CA (US); Masahiko Hoshijima, La Jolla, CA (US)

Correspondence Address: BROWN, MARTIN, HALLER & MCCLAIN LLP, 1660 UNION STREET, SAN DIEGO, CA, 92101-2926, US

(21) Appl. No.: 10/136,819

(22) Filed: Apr. 30, 2002

Related U.S. Application Data
(60) Provisional application No. 60/287,423, filed on Apr. 30, 2001.

(51) Int. Cl. A61K 48/00; C12N 15/74; C12N 15/85

(52) U.S. Cl. 514/44; 435/455; 435/320.1

(57) ABSTRACT

The invention is a non-viral vesicle vector for the delivery of nucleic acid to various cardiac cell types. The vesicle vector contains the hepatitis B envelope protein wherein at least part of the liver targeting sequence is deleted and replaced with a specific cardiac cell targeting sequence. The targeting sequence may be derived from viruses that have the natural tropism desired (e.g. adenovirus type 5 knob protein for cardiomyocyte delivery) or mammalian sequences (e.g. endothelin-1 for vascular endothelial cell delivery). The vesicle vector contains an expression construct for the expression of therapeutic genes in cardiac tissues.

### CROSS-REFERENCES TO RELATED APPLICATIONS

This application claims the benefit of priority of U.S. provisional application Serial No. 60/287,423 filed Apr. 30, 2001 which is incorporated herein by reference in its entirety.

### SEQUENCE LISTING

A sequence listing is submitted herewith under 35 C.F.R. §1.821 and is incorporated herein by reference.

### BACKGROUND OF THE INVENTION

The utility of gene delivery vectors for gene therapy is limited by the nonselective nature in which the vectors, either non-viral or viral, interact with the cell surface, resulting in transduction of numerous cell types in addition to the target cells. Much effort has been devoted to understand the mechanisms of viral targeting to different cell types. The information derived from these studies is now being exploited to select viruses that have the desired natural tropism or to modify viral targeting signals to redirect viruses to the cell type of choice.

Methods to redirect the targeting of viral vectors include the use of bi-specific antibodies (Wickham et al, 1996). Adenoviral vectors with a fiber protein modified to contain the antigenic FLAG peptide sequence were prepared. A bispecific antibody comprised of a monoclonal antibody to the FLAG epitope and a monoclonal antibody to a cell type specific surface antigen (e.g. αᵥ integrin for targeting endothelial and smooth muscle cells) was bound to the adenovirus to target it to αᵥ integrin expressing cells. Transduction of specific cell types was greatly increased by the use of this method, however the method is sufficiently cumbersome to make it impractical for use in gene therapy. Such a method also requires knowledge of a specific cell surface receptor to which a portion of the bispecific antibody can be directed.

Subsequently, viral particles were modified directly to contain targeting sequences such as integrin binding peptide sequences (Dmitriev et al., 1998). The H1 loop of the adenovirus fiber knob was modified to contain the RGD integrin binding sequence. The modified vector was then able to transduce primary tumor cells with greater efficiency. Phage display screening methods have been used to select peptides for incorporation into adenovirus fiber protein to allow for targeting to cell types with unknown cell surface receptors (Nicklin et al., 2000). Insertion of sequences to promote specific targeting can be coupled with mutation of natural targeting sequences to increase the tissue specificity of the virus.

Although the methods above have helped to resolves some tissue specificity problems, gene therapy protocols are also limited by the absence of a high efficiency gene delivery vector that can delivery large constructs, be produced on a sufficient scale for use and is safe for administration to humans. Adenovirus, adeno-associated virus (AAV) and the majority of retroviruses have a small capacity for heterologous nucleic acid sequence (i.e. 4.5 Kb for AAV and approximately 8 Kb for conventional adenovirus) which is insufficient for a number of applications. Preparation of high titer viral stocks is difficult and laborious. Viral vectors also contain a portion of the viral genome which is not ideal for safety considerations, especially with the use of retroviruses.

The development of good gene delivery vectors would be a boon to a number of medical fields, including cardiology. Cardiovascular gene therapy could be used for the treatment of ischemic diseases by delivery of angiogenic factors, restenosis after angioplasty and stent implantation, and to treat atherogenesis and thrombogenesis. Heart failure could be prevented or treated by the transfer of genes that would correct problems with calcium handling. The major limitation of cardiac gene therapy is the lack of a good gene delivery vector with high gene transfer efficiency and minimal side effects.

Striated muscle tissue is considered amenable for gene transfer because of natural tissue selectivity if adenovirus and AAV, because of high expression of Coxsackie-adenovirus receptor (CAR), the adenovirus receptor, in this tissue. Type 2 AAV associates with membrane associated heparin sulfate proteoglycan (HSPG). In skeletal muscle, type 2 AAV predominantly transduces slow myofibers, which are very similar to cardiac muscle cells. Several experimental attempts of gene transfer therapy to the myocardium have been undertaken. Recently highly efficient gene delivery to cardiac tissue using adenovirus and AAV was recently achieved. Both viral vectors can be used to transfer genes to non-dividing cells, including rod shaped adult cardiomyocytes. However, adenovirus and AAV vectors have been found to be inefficient in transduction of vascular tissues. Attempts to deliver genes to vascular cells with retroviral and non-viral vectors have been similarly disappointing.

Studies are being undertaken to increase the specificity of non-viral vectors. A novel, liver-specific vesicle vector expressing modified surface proteins of the hepatitis B virus was recently described by Yamada et al (2001 a). The vesicles containing the hepatitis B membrane proteins are generated by the methods well known to those skilled in the art (Kuroda et al, 1992, and Yamada et al., 2001b, incorporated herein by reference). Briefly, a modified hepatitis B envelope (env) L protein, containing the pre-S1+pre-S2+S peptides, can be effectively generated in yeast by fusing the coding sequence for the chicken lysozyme signal sequence in frame to the beginning of the coding sequence for the modified env L protein (SEQ ID 1). The signal sequence directs the insertion of the proteins into the endoplasmic reticulum during translation. Protein rich vesicles bud from the endoplasmic reticulum and accumulate in the cytoplasm of the yeast cell. The vesicles are composed of lipid bilayers derived from the ER with the modified env L protein as the major protein component. Particles formed by this method are very stable and can be easily purified through repetitive cesium chloride and sucrose gradients by methods well known to those skilled in the art.

Plasmid DNA into the env L containing particles by electroporation (Yamada et al. 2001 a). Such DNA containing particles were demonstrated to facilitate entry of the DNA specifically into liver cells both in culture and upon systemic administration to nude mice. Yamada et al. (2001 a) suggested that such a vesicle vector could be used for tissue specific delivery of nucleic acid and other compounds to the any tissue by altering the tissue targeting sequence exposed on the surface of the vesicle.

### SUMMARY OF THE INVENTION

The invention is the modification of the hepatitis B env L vesicle vector for delivery of genes to cardiac cells. Targeting sequences from a number of sources including targeting sequences from vectors with a natural tropism for various types of cardiac cells and sequences of endogenous ligands that bind to surface proteins present on cardiac cells. For example, two peptide sequences from coat protein of viral vectors that are widely used in muscle gene delivery can be used for delivery to cardiomyocytes. The C-terminal knob-domain of Ad5 fiber protein is known to bind CAR, which is highly expressed on the surface of cardiomyocytes. The loop IV region of VP3 capsid protein of AAV binds to heparin sulfate proteoglycans on the surface of cardiomyocytes. For targeting cells throughout the circulatory system including cardiomyocytes, smooth muscle cells and endothelial cells, αᵥ integrin binding RGD peptides or poly-lysine motifs (pK7) are incorporated. To selectively target vascular endothelial cells, peptides including vascular endothelial growth factor (VEGF), platelet-endothelial cell adhesion molecule-1 (PCAM-1), angiopoietin-1 and -2, any of the family of ephrins (e.g. ephrin-A1), L-seelctin, CD34, Lfa-1 and Mac-1 are used. Various vasotropic peptides including angiotensin 1 and 11 and endothelin-1 are used to broadly target the cardiovascular system. The targeting sequence is selected to infect the species of interest.

The unique feature of the invention is the use of the tissue selective non-viral vector to deliver gene expression cassettes, proteins or other agents to cardiovascular tissues. Targeting the non-viral gene delivery vesicles via peptides known to specifically bind the surface of specific cardiac and vascular cell types provides a method to direct highly efficient gene delivery. The safety of such non-viral vectors in humans is already established as the vesicle vector has long been used for the hepatitis B virus vaccine. This is in contrast to viral gene transfer vectors which include much of the genome of the virus from which they were derived. Production of the vesicles is relatively easy as compared to production of high titer virus stocks. The vesicles are highly stable and can be produced in large quantities making them ideal for gene therapy.

The invention is a method for the treatment of cardiovascular disease comprising the administration of the non-viral vector of the invention preferably containing a gene expression construct, possibly in conjunction with other agents. The expression construct may be single or double stranded DNA containing any of a number of promoters including, but not limited to general (e.g. cytomegalovirus, Rous sarcoma virus) and tissue specific (e.g.myosin light chain 2v, cardiac ankirin repeat, ANF and BNP) promoters. The construct may contain additional regulatory elements including, but not limited to enhancers, introns, poly A sequences, RNA targeting sequences. Sequences to promote replication of the plasmid including SV40 origin of replication can be included. Inverted terminal repeat (ITR) sequences from AAV can be included in the construct to promote expression cassette stability or to enhance integration into the host DNA with the AAV Rep protein. In lieu of ITR sequences, eukaryotic DNA transposon/transposases systems can be used to promote integration. The non-viral vesicle vector detailed is for delivery of therapeutic constructs to humans. However, it is well within the skill of those in the art to modify the vesicle vector of the instant invention for the use in any of a number of animals, especially mammals.

Gene therapy may be used alone to deliver growth factors (e.g. delivery of VEGF to promote angiogenesis) or to enhance expression of gene products in the cell (e.g. delivery of sarcoplasmic reticulum Ca²⁺-ATPase (SERCA) or mutated forms of phospholamban to restore Ca²⁺ normal contractility). Alternatively, gene therapy can be used in conjunction with other therapies (e.g. delivery of antisense cdc2 and antisense proliferating cell nuclear antigen (PCNA) to prevent restenosis after balloon angioplasty). As the vesicle vector contains cell specific targeting signals, it can be delivered intravenously or intra-arterially rather than by more invasive methods (e.g. direct cardiac injection).

### DETAILED DESCRIPTION AND PREFERRED EMBODIMENTS

The invention is a vesicle vector for the treatment of cardiac disease comprising a natural lipid vesicle preferably produced in yeast or Sf9 insect cells containing hepatitis B env L protein modified to contain a cardiac targeting sequence in the S1 region such that the targeting sequence is exposed on the surface of the vesicle and an nucleic acid construct inside the vesicle for the expression of a therapeutic nucleotide sequence or gene for cardiac cells. The vesicles are prepared by the vaccine production method of Kuroda (1992) further refined by Yamada (2001 b). Briefly, the hepatitis B env L protein is composed of three regions: the 108- or 119-residue pre-S1 region involved in the direct interaction with hepatocytes, the 55-residue pre-S2 region associated with the polymerized albumin-mediated interaction and the major 226-residue S-protein region. Attempts to produce L protein in various eukaryotic cells had been unsuccessful, probably due to the presence of the N-terminus of the pre-S1 peptide. The coding sequence of the N-terminus of the L protein was replaced by a chicken lysosome signal sequence to direct the translocation of the N-terminus through the endoplasmic reticulum (ER). The chimeric sequence was inserted into a yeast (S. cerevisiae) expression vector and inserted into yeast using a standard transformation protocol. The chimeric L-protein was produced in abundance, up to 42% of the total yeast protein, and was determined to be properly inserted into the membrane. Vesicles budded off of the ER to form 23 nm spherical and filamentous particles containing the protein in the membrane of the vesicles. The yeast cells were disrupted with glass beads to release the vesicles. Vesicles were purified by serial rounds of discontinuous cesium and sucrose gradients. Production and purification of vesicles from insect cells would be performed in a similar method. A crude membrane fraction could be prepared as with the yeast cells, by homogenization and differential centrifugation. The fraction can be loaded onto cesium or sucrose gradients as with the yeast extract for purification of vesicles. The methods are amenable to inexpensive, large scale production of vesicles which is necessary for gene transfer. Vesicles are stable for long term storage at a low temperature but are unstable upon repeated freeze-thaw cycles.

A number of cardiac targeting sequences are available for use in the vesicle vector of the invention. The selection of a specific sequence is dependent upon the cardiac tissue to be targeted. For example, viral targeting sequences from type 5 adenovirus and AAV can be used for targeting cardiomyocytes. Vascular endothelial cells are more efficiently targeted by the use of natural peptide ligands such as vascular endothelial growth factor (VEGF), platelet-endothelial cell adhesion molecule (PCAM-1), angiopoietin-1 and -2, ephrins (such as ephrin-A1 precursor), L-selectin, CD34, LFA-1 and Mac-1. RGD peptides, preferably cyclic, have a broad specificity for a number of vascular tissues (Koivunen et al, 1995, incorporated herein by reference) as do poly-lysine motifs. Target cardiac tissues and ligands for integration into the vesicle vector of the invention are listed below. Sequences listed are human targeting sequences for delivery of agents to human tissues. It is understood that species appropriate targeting sequences can be selected to target the non-viral vesicle vector in various organisms (e.g. mouse sequences could be incorporated for delivery of agents to mouse tissues). Additionally, the full length coding sequence of the targeting protein need not be incorporated into the hepatitis B surface protein. Methods to determine the essential factors for targeting to the tissue of interest are well known (e.g. phage panning, affinity chromatography, far western blots). Such selections are routine in the art and should not be considered a limitation of the invention.

| Cell type | Ligand |
|---|---|
| broad specificity, | angiotensin (SEQ ID 2) |
| cardiovascular | endothelin-1 (SEQ ID 3) |
| | endothelin-2 (SEQ ID 4) |
| cardiomyocyte | adenovirus type 5 fiber protein (SEQ ID 5, nt |
| | 385-581 or portion thereof) |
| | AAV 2 coat protein sequences, VP1 loop IV |
| | domain (SEQ ID 6, nt 416-646 or portion thereof) |
| | coxsackie virus B3, VP1 (SEQ ID 7) |
| | coxsackie virus receptor (SEQ ID 8) |
| vascular cells | RGD peptides, preferably cyclic (SEQ ID 9) |
| | polylysine motifs |
| vascular endothelial | VEGF (SEQ ID 10) |
| cells | PCAM-1 (SEQ ID 11) |
| | angiopoietin-1 (SEQ ID 12) |
| | angiopoietin-2 (SEQ ID 13) |
| | ephrins, e.g. ephrin-A1 (SEQ ID 14) |
| | L-selectin (SEQ ID 15) |
| | CD34 (SEQ ID 16) |
| | LFA-1 (SEQ ID 17) |
| | Mac-1 (SEQ ID 18) |

The vesicle vectors can be used for the delivery of any nucleic acid construct, single- or double-stranded DNA or RNA, to the cardiac tissue. The nucleic acid sequence to be delivered would depend on the disease state and the tissue to which the gene is delivered. Potential therapies include long term expression of gene products to replace or enhance expression of proteins for the treatment of heart failure such as mutant forms of phospholamban (see WO 00/25804, incorporated herein by reference) SERCA-2, G-protein coupled receptors, G-protein coupled receptor modifier or -adrenergic receptor (β-AR) to increase cardiac contractility. Arrhythmia can be treated by expression of potassium channels and their associated molecules. Reperfusion injury can by treated by expression of superoxide dismutase (SOD) or nitric oxide synthase (NOS). Atherosclerosis can be treated by expression of negative cell cycle regulators or a lipoprotein receptor such as low density lipoprotein (LDL) receptor. Alternatively, antisense oligonucleotides can be produced short term to inhibit expression of cdc2 and PCNA of can be produced to inhibit restenosis after balloon angioplasty.

The construct may optionally contain additional regulatory and enhancer elements to modulate gene expression, intron and poly-A sequences to promote RNA processing and gene expression, RNA targeting sequences, AAV-ITR or eukaryotic transposon sequences to promote stabilization of expression cassettes and integration into the host genome and viral origin of replication sequences to promote amplification of the plasmid in host cells. Such sequences are well known to those skilled in the art. The number of elements that can be inserted into the nucleic acid construct as the size is not limited by the requirements of a viral genome as is the case with many gene transfer protocols.

Any of a number of promoter sequences are known to be functional in cardiac cells. These include both non-tissue specific promoters such as CMV, RSV, ubiquitin, chicken β-actin and elongation factor (EF)-1α; and tissue specific promoters such as myosin light chain 2v, CARP, ANF and BNP for cardiomyocytes; SM22 for smooth muscle cells; and Fit-1 (VEGFR-1), Flk-1 (VEGFR-2), endothelial type nitric oxide synthase (eNOS) and endothelin.

AAV-ITR sequences may be incorporated into the construct flanking all of the coding and regulatory sequences, other than any origins of replication. The AAV-ITR sequences have been demonstrated to increase the stability of transferred constructs in gene therapy protocols. Alternatively, the AAV-ITR sequences may enhance integration into the human genome at a specific site with the cooperation of the AAV-Rep protein, which may be supplied by incorporation into the vesicles with the nucleic acid construct or by expression cassettes packaged into the same vesicle.

Eukaryotic transposon sequences can be incorporated into the construct flanking all of the coding sequences and regulatory elements, similar to the AAV-ITR sequences. Transposase to promote integration may be expressed from the same expression cassette or from a separate expression cassette packaged into the same vesicle.

The coding sequence incorporated into the expression cassette or the agent to be delivered will be dependent on the disease to be treated, as discussed above. The specific contents of the non-viral vesicle vector is not a limitation of the instant invention.

In a preferred embodiment, the nucleic acid construct of the invention is introduced into the vesicles by electroporation. The nucleic acid construct is mixed thoroughly with the vesicles, brought to a final volume in water and transferred to an electroporation cuvette. Voltage and resistance vary widely depending on the size (gap length) of the cuvette and the volume of material in the cuvette. Such parameters can be readily modified by methods well known to those skilled in the art to result in maximum transfer of nucleic acid into vesicles with minimum destruction of vesicles.

Alternatively the nucleic acid may be introduced into the vesicle by fusion with nucleic acid containing liposomes by methods well known to those skilled in the art (Dzau et al, 1996). The construct of the invention is encapsulated into liposomes prepared by vortexing. Liposomes may be composed of known phospholipids and membrane components (e.g. phosphatidyl-choline, cholesterol) or of commercially available proprietary mixtures of membrane components (e.g. Lipofectamine from Gibco-BRL). Nucleic acid encapsulated in liposomes will fuse with the yeast or insect cell derived vesicles upon incubation at 37° C. for 10-30 minutes.

The nucleic acid or protein containing non-viral vesicle vectors of the invention are administered to the individual intravenously or intraarterially. To increase delivery, the vesicle vector can be administered directly adjacent to the heart. Such an application would be most common in conjunction with surgery. The individual is monitored on regular intervals for the expression of the gene products or for phenotypic recovery. The amount of the non-viral vesicle to be administered would depend on the strength of the promoter, integration sequences, number of plasmids per vesicle and a number of other considerations well know to those skilled in the art. As methods for monitoring the state of health of individuals are well known, an effective dose can be readily determined.

### EXAMPLE 1

Delivery of SERCA-2 to caridomyocytes using a viral targeting sequence. The coding sequence of the S1 portion of the hepatitis B env L protein containing the chicken lysosome signal sequence was modified to contain all or part of the knob domain of adenovirus type 5 (amino acids 385-581) (SEQ ID 2) such that the cardiomyocyte binding domain is exposed on the exterior surface of the vesicle vector. Methods for modifying nucleic acid sequences are well known to those skilled in the art. The coding sequence for the modified env L protein is inserted into an appropriate expression vector and transformed into yeast. Vesicles are purified from the yeast by the method of Kuroda (1992) and Yamada (2001 b). A plasmid vector for the expression of pseudophosphorylated mutants of phospholamban or SERCA-2 in cardiomyocytes is generated containing the coding sequence for SERCA-2 driven by a CMV promoter. The coding sequence and promoter are flanked by AAV-ITR sequences. The plasmid is introduced into the vesicles by electroporation. The vesicles are administered intravenously to the patient. Cardiac function is monitored by methods well known to those skilled in the art (e.g. blood pressure, EKG) to determine the efficacy of the gene therapy protocol.

### EXAMPLE 2

Delivery of VEGF to vascular endothelial cells using an integrin targeting sequence. The coding sequence of the S1 portion of the hepatitis B env L protein containing the chicken lysosome signal sequence is modified to contain any of a number of αᵥ integrin RGD binding motifs (Koivunen et al, 1995, incorporated herein by reference) such that the RGD sequence is exposed on the exterior surface of the vesicle vector. The RGD sequence preferably contains cysteine residues to allow for the formation of disulfide bonds to form a cyclic peptide (e.g. ACDCRGDCFCG) (SEQ ID 9). Methods for modifying nucleic acid sequences are well known to those skilled in the art. The coding sequence for the modified env L protein is inserted into an appropriate expression vector and transformed into yeast. Vesicles are purified from the yeast by the method of Kuroda (1992) and Yamada (2001 b). A plasmid vector for the expression of VEGF in vascular endothelial cells is generated containing the coding sequence for VEGF driven by an RSV promoter. The coding sequence and promoter are flanked by AAV-ITR sequences. The plasmid is introduced into the vesicles by electroporation. The vesicles are administered intravenously to the patient. VEGF stimulated angiogenesis is monitored using imaging methods known to those skilled in the art.

Although an exemplary embodiment of the invention has been described above by way of example only, it will be understood by those skilled in the field that modifications may be made to the disclosed embodiment without departing from the scope of the invention, which is defined by the appended claims.

### REFERENCES

Dmitriev, I et al. (1998) An adenovirus vector with genetically modified fibers demonstrates expanded tropism via utilization of a coxsackievirus and adenovirus receptor-independent cell entry mechanism. J. Virol. 72:9706-9713.

Koivunen, E., et al (1995) Phage libraries displaying cyclic peptides with different ring sizes: ligand specificities of the RGD-directed integrins. Biotechnology 13:265-70.

Kuroda, S. et al (1992) Hepatitis B virus envelope L protein particles. J. Biol. Chem. 267:1953-1961.

Nicklin, S. A. (2000) Selective targeting of gene transfer to vascular endothelial cells by use of peptides isolated by phage display. Circulation 102:231-237.

Wickham, T. J. et al. (1996) Targeted adenovirus gene transfer to endothelial and smooth muscle cells using bispecific antibodies. J. Virol. 70:6831-6838.

Yamada, T. et al (2001 a) A new pinpoint gene delivery system using genetically engineered hepatitis B virus envelope L particles. Molecular Biology and New Therapeutic Strategies: Cancer Research in the 21^{st} Century. 5^{th} Joint Conference of the American Association for Cancer Research and the Japanese Cancer Association. Hawaii, USA, Feb. 12-16, 2001.

Yamada. T. et al (2001 b) Physiochemical and immunological characterization of hepatitis B virus envelope particles exclusively consisting of the entire L (pre-S1+pre-S2+S) protein. Vaccine 19:3154-3163.

Sequence Accession Numbers K02215; XM_{—}043856; NM_{—}001956; M18369; AF043303; M33854; NM_{—}001338; NP_{—}003377; M28526; AAB50557; XP_{—}034835; NM004428; X117519; M81104; Y00796; J03925 all incorporated herein by reference.

We claim:

1. A non-viral vector comprising:
a vesicular membrane with hepatitis B envelope (env) protein containing a cardiac targeting sequence exposed on the surface of the vesicle and
a nucleic acid construct comprising a nucleotide sequence for cardiovascular gene therapy and a promoter sequence functional in cardiovascular cells.

2. The vesicle vector of claim 1, wherein the env protein contains mutations to reduce antigenicity.

3. The vesicle vector of claim 1, wherein the expression construct is DNA.

4. The vesicle vector of claim 1, wherein the expression construct is double stranded plasmid DNA.

5. The vesicle vector of claim 1, wherein the expression construct is RNA.

6. The vesicle vector of claim 1, wherein the promoter is a non-tissue specific promoter.

7. The vesicle vector of claim 6, wherein the viral promoter is selected from the group consisting of cytomegalovirus promoter, Rous sarcoma virus promoter ubiquitin promoter, chicken β-actin promoter and elongation factor 1α promoter.

8. The vesicle vector of claim 1, wherein the promoter is a cardiomyocyte specific promoter.

9. The vesicle vector of claim 8, wherein the cardiomyocyte specific promoter is selected from the group consisting of myosin light chain 2v promoter, cardiac ankyrin repeat protein (CARP) promoter, ANF promoter and BNP promoter.

10. The vesicle vector of claim 1, wherein the promoter is a smooth muscle cell specific promoter.

11. The vesicle vector of claim 10, where the smooth muscle cell specific promoter is SM22 promoter.

12. The vesicle vector of claim 11, wherein the promoter is an endothelial cell specific promoter.

13. The vesicle vector of claim 12, wherein the endothelial cell specific promoter is selected from the group consisting of Flt-1 promoter, Flk-1 promoter, endothelial type nitric oxide synthase promoter and endothelin promoter.

14. The vesicle vector of claim 1, wherein the expression construct comprises inverted terminal repeat sequences from adeno-associated virus (AAV-ITR).

15. The vesicle vector of claim 1, wherein the expression construct comprises eukaryotic transposon and transposase elements.

16. The vesicle vector of claim 1, wherein the cardiovascular targeting sequence comprises a viral protein sequence.

17. The vesicle vector of claim 1, wherein the cardiovascular targeting sequence comprises a natural ligand for a receptor on cardiovascular cells.

18. A non-viral vesicle vector comprising:
a vesicular membrane with hepatitis B env protein exposed on the vesicle surface and
a protein for treatment of cardiovascular disease.

19. The vesicle vector of claim 18, wherein the env protein contains mutations to reduce antigenicity.

20. A method for treatment of cardiac disease comprising:
intravenous administration to an individual with cardiac disease a non-viral vesicle vector comprising a vesicular membrane with hepatitis B env protein with a cardiac targeting sequence exposed on the vesicle surface and
a nucleic acid construct comprising a nucleotide sequence for cardiac gene therapy and a promoter sequence functional in cardiac cells
monitoring the individual for amelioration of disease.


SEQUENCE LISTING (18 sequences)

SEQ ID NO: 1 - DNA, 1286 bp, Hepatitis B virus
gtcgagtataaaaacaatgagatctttgttgatcttggttttgtgtttcttgccattggctgctttgggtaaggttcgacaaggcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcaatggccagaggcaaatcaggtaggagcgggagcattcgggccagggttcaccccaccacacggcggtcttttggggtggagccctcaggctcagggcatattgacaacagtgccagcagcacctcctcctgcctccaccaatcggcagtcaggaagacagcctactcccatctctccacctctaagagacagtcatcctcaggccatgcagtggaattccacaacattccaccaagctctgctagatcccagagtgaggggcctatattttcctgctggtggctccagttccggaacagtaaaccctgttccgactactgcctcacccatatctggggaccctgcaccgaacatggagaacacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaaccaccagcacggggccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttaaattgaattgaattgaa

SEQ ID NO: 2 - DNA, 2099 bp, Homo sapiens
aagaagctgccgttgttctgggtactacagcagaagggtatgcggaagcgagcaccccagtctgagatggctcctgccggtgtgagcctgagggccaccatcctctgcctcctggcctgggctggcctggctgcaggtgaccgggtgtacatacaccccttccacctcgtcatccacaatgagagtacctgtgagcagctggcaaaggccaatgccgggaagcccaaagaccccaccttcatacctgctccaattcaggccaagacatcccctgtggatgaaaaggccctacaggaccagctggtgctagtcgctgcaaaacttgacaccgaagacaagttgagggccgcaatggtcgggatgctggccaacttcttgggcttccgtatatatggcatgcacagtgagctatggggcgtggtccatggggccaccgtcctctccccaacggctgtctttggcaccctggcctctctctatctgggagccttggaccacacagctgacaggctacaggcaatcctgggtgttccttggaaggacaagaactgcacctcccggctggatgcgcacaaggtcctgtctgccctgcaggctgtacagggcctgctagtggcccagggcagggctgatagccaggcccagctgctgctgtccacggtggtgggcgtgttcacagccccaggcctgcacctgaagcagccgtttgtgcagggcctggctctctatacccctgtggtcctcccacgctctctggacttcacagaactggatgttgctgctgagaagattgacaggttcatgcaggctgtgacaggatggaagactggctgctccctgatgggagccagtgtggacagcaccctggctttcaacacctacgtccacttccaagggaagatgaagggcttctccctgctggccgagccccaggagttctgggtggacaacagcacctcagtgtctgttcccatgctctctggcatgggcaccttccagcactggagtgacatccaggacaacttctcggtgactcaagtgcccttcactgagagcgcctgcctgctgctgatccagcctcactatgcctctgacctggacaaggtggagggtctcactttccagcaaaactccctcaactggatgaagaaactgtctccccggaccatccacctgaccatgccccaactggtgctgcaaggatcttatgacctgcaggacctgctcgcccaggctgagctgcccgccattctgcacaccgagctgaacctgcaaaaattgagcaatgaccgcatcagggtgggggaggtgctgaacagcattttttttgagcttgaagcggatgagagagagcccacagagtctacccaacagcttaacaagcctgaggtcttggaggtgaccctgaaccgcccattcctgtttgctgtgtatgatcaaagcgccactgccctgcacttcctgggccgcgtggccaacccgctgagcacagcatgaggccagggccccagaacacagtgcctggcaaggcctctgcccctggcctttgaggcaaaggccagcagcagataacaaccccggacaaatcagcgatgtgtcacccccagtctcccaccttttcttctaatgagtcgactttgagctggaaagcagccgtttctccttggtctaagtgtgctgcatggagtgagcagtagaagcctgcagcggcacaaatgcacctcccagtttgctgggtttattttagagaatgggggtggggaggcaagaaccagtgtttagcgcgggactactgttccaaaaagaattccaaccgaccagcttgtttgtgaaacaaaaaagtgttcccttttcaagttgagaacaaaaattgggttttaaaattaaagtatacatttttgcattgccttcggtttgtatttagtgtcttgaatgtaagaacatgacctccgtgtagtgtctgtaataccttagttttttccacagatgcttgtgatttttgaacaatacgtgaaagatgcaagcacctgaatttctgtttgaatgcggaacaatagctggttatttctcccttgtgttagtaataaacgtcttgccac

SEQ ID NO: 3 - DNA, 1250 bp, Homo sapiens
ggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagcacgttgttccgtatggacttggaagccctaggtccaagagagccttggagaatttacttcccacaaaggcaacagaccgtgaaaatagatgccaatgtgctagccaaaaagacaagaagtgctggaatttttgccaagcaggaaaagaactcagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtcggagaccatgagaaacagcgtcaaatcatcttttcatgatcccaagctgaaaggcaagccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattc

SEQ ID NO: 4 - DNA, 1240 bp, Homo sapiens
ccagcttaatagcaggacgctggcaacaggcactccctgctccagtccagcctggcgctccaccgccgctatggtctccgtgcctaccacctggtgctccgttgcgctagccctgctcgtggccctgcatgaagggaagggccaggctgctgccaccctggagcagccagcgtcctcatctcatgcccaaggcacccaccttcggcttcgccgttgctcctgcagctcctggctcgacaaggagtgcgtctacttctgccacttggacatcatctgggtgaacactcctgaacagacagctccttacggcctgggaaacccgccaagacgccggcgccgctccctgccaaggcgctgtcagtgctccagtgccagggaccccgcctgtgccaccttctgccttcgaaggccctggactgaagccggggcagtcccaagccggaagtcccctgcagacgtgttccagactggcaagacaggggccactacaggagagcttctccaaaggctgagggacatttccacagtcaagagcctctttgccaagcgacaacaggaggccatgcgggagcctcggtccacacattccaggtggaggaagagatagtgtcgtgagctggaggaacattgggaaggaagcccgcggggagagaggaggagagaagtggccagggcttgtggactctctgcctgcttcctggaccggggccttggtcccagacagctggacccatttgccaggattggcacaagctccctggtgagggagcctcgtccaaggcagttctgtgtcctcgcactgcccagggaagccctcggcctccagactgcggagcagcctccagtgctggctgctggcccacagctctgctggaagaactgcatggggagtacattcatctggaggctgcgtcctgaggagtgtcctgtctgctgggctacaaaccaggagcaaccgtgcagccacgaacacgcatgcctcagccagccctggagactggatggctcccctgaggctggcatcctggctggctgtgtcctctccagctttccctccccagagttcttgcaccctcattccctcgggaccctcccagtgagaagggcctgctctgcttttcctgtctgtatataacttatttgccctaagaactttgagaatcccaattatttattttaatgtattttttagaccctctatttacctgcgaacttgtgtttataataaatgaggaaacat

SEQ ID NO: 5 - DNA, 2530 bp, Adenovirus type 5
gttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccggg

SEQ ID NO: 6 - DNA, 4679 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 7 - DNA, 7399 bp, Coxsackievirus B3
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagctcaagtatcaacgcaaaagactggggcacatgagaccaggctgaatgctagcggcaattccatcattcactacacaaatattaattattacaaggatgccgcatccaactcagccaatcggcaggatttcactcaagacccgggcaagttcacagaaccagtgaaagatatcatgattaaatcactaccagctctcaactcccccacagtagaggagtgcggatacagtgacagggcgagatcaatcacattaggtaactccaccataacgactcaggaatgcgccaacgtggtggtgggctatggagtatggccagattatctaaaggatagtgaggcaacagcagaggaccaaccgacccaaccagacgttgccacatgtaggttctatacccttgactctgtgcaatggcagaaaacctcaccaggatggtggtggaagctgcccgatgctttgtcgaacttaggactgtttgggcagaacatgcagtaccactacttaggccgaactgggtataccgtacatgtgcagtgcaatgcatctaagttccaccaaggatgcttgctagtagtgtgtgtaccggaagctgagatgggttgcgcaacgctagacaacaccccatccagtgcagaattgctggggggcgatacggcaaaggagtttgcggacaaaccggtcgcatccgggtccaacaagttggtacagagggtggtgtataatgcaggcatgggggtgggtgttggaaacctcaccattttcccccaccaatggatcaacctacgcaccaataatagtgctacaattgtgatgccatacaccaacagtgtacctatggataacatgtttaggcataacaacgtcaccctaatggttatcccatttgtaccgctagattactgccctgggtccaccacgtacgtcccaattacggtcacgatagccccaatgtgtgccgagtacaatgggttacgtttagcagggcaccagggcttaccaaccatgaatactccggggagctgtcaatttctgacatcagacgacttccaatcaccatccgccatgccgcaatatgacgtcacaccagagatgaggatacctggtgaggtgaaaaacttgatggaaatagctgaggttgactcagttgtcccagtccaaaatgttggagagaaggtcaactctatggaagcataccagatacctgtgagatccaacgaaggatctggaacgcaagtattcggctttccactgcaaccagggtactcgagtgtttttagtcggacgctcctaggagagatcttgaactattatacacattggtcaggcagcataaagcttacgtttatgttctgtggttcggccatggctactggaaaattccttttggcatactcaccaccaggtgctggagctcctacaaaaagggttgatgctatgcttggtactcatgtaatttgggacgtggggctacaatcaagttgcgtgctgtgtataccctggataagccaaacacactaccggtttgttgcttcagatgagtataccgcagggggttttattacgtgctggtatcaaacaaacatagtggtcccagcggatgcccaaagctcctgttacatcatgtgtttcgtgtcagcatgcaatgacttctctgtcaggctattgaaggacactcctttcatttcgcagcaaaactttttccagggcccagtggaagacgcgataacagccgctatagggagagttgcggataccgtgggtacagggccaaccaactcagaagctataccagcactcactgctgctgagacgggtcacacgtcacaagtagtgccgggtgacactatgcagacacgccacgttaagaactaccattcaaggtccgagtcaaccatagagaacttcctatgtaggtcagcatgcgtgtactttacggagtataaaaactcaggtgccaagcggtatgctgaatgggtattaacaccacgacaagcagcacaacttaggagaaagctagaattctttacctacgtccggttcgacctggagctgacgtttgtcataacaagtactcaacagccctcaaccacacagaaccaagatgcacagatcctaacacaccaaattatgtatgtaccaccaggtggacctgtaccagataaagttgattcatacgtgtggcaaacatctacgaatcccagtgtgttttggaccgagggaaacgccccgccgcgcatgtccataccgtttttgagcattggcaacgcctattcaaatttctatgacggatggtctgaattttccaggaacggagtttacggcatcaacacgctaaacaacatgggcacgctatatgcaagacatgtcaacgctggaagcacgggtccaataaaaagcaccattagaatctacttcaaaccgaagcatgtcaaagcgtggatacctagaccacctagactctgccaatacgagaaggcaaagaacgtgaacttccaacccagcggagttaccactactaggcaaagcatcactacaatgacaaatacgggcgcatttggacaacaatcaggggcagtgtatgtggggaactacagggtggtaaatagacatctagctaccagtgctgactggcaaaactgtgtgtgggaaagttacaacagagacctcttagtgagcacgaccacagcacatggatgtgatattatagccagatgtcagtgcacaacgggagtgtacttttgtgcgtccaaaaacaagcactacccaatttcgtttgaaggaccaggtctagtagaggtccaagagagtgaatactaccccaggagataccaatcccatgtgcttttagcagctggattttccgaaccaggtgactgtggcggtatcctaaggtgtgagcatggtgtcattggcattgtgaccatggggggtgaaggcgtggtcggctttgcagacatccgtgatctcctgtggctggaagatgatgcaatggaacagggagtgaaggactatgtggaacagcttggaaatgcattcggctccggctttactaaccaaatatgtgagcaagtcaacctcctgaaagaatcactagtgggtcaagactccatcttagagaaatctctaaaagccttagttaagataatatcagccttagtaattgtggtgaggaaccacgatgacctgatcactgtgactgccacactagcccttatcggttgtacctcgtccccgtggcggtggctcaaacagaaggtgtcacaatattacggaatccctatggctgaacgccaaaacaatagctggcttaagaaatttactgaaatgacaaatgcttgcaagggtatggaatggatagctgtcaaaattcagaaattcattgaatggctcaaagtaaaaattttgccagaggtcagagaaaaacacgagttcctgaacagacttaaacaactccccttattagaaagtcagatcgccacaatcgagcagagcgcgccatcccaaagtgaccaggaacaattattttccaatgtccaatactttgcccactattgcagaaagtacgctcccctctacgcagctgaagcaaagagggtgttctcccttgagaagaagatgagcaattacatacagttcaagtccaaatgccgtattgaacctgtatgtttgctcctgcacgggagccctggtgccggcaagtcggtggcaacaaacttaattggaaggtcgcttgctgagaaactcaacagctcagtgtactcactaccgccagacccagatcacttcgacggatacaaacagcaggccgtggtgattatggacgatctatgccagaatcctgatgggaaagacgtctccttgttctgccaaatggtttccagtgtagattttgtaccacccatggctgccctagaagagaaaggcattctgttcacctcaccgtttgtcttggcatcgaccaatgcaggatctattaatgctccaaccgtgtcagatagcagagccttggcaaggagatttcactttgacatgaacatcgaggttatttccatgtacagtcagaatggcaagataaacatgcccatgtcagtcaagacttgtgacgatgagtgttgcccggtcaattttaaaaagtgctgccctcttgtgtgtgggaaggctatacaattcattgatagaagaacacaggtcagatactctctagacatgctagtcaccgagatgtttagggagtacaatcatagacatagcgtggggaccacgcttgaggcactgttccagggaccaccagtatacagagagatcaaaattagcgttgcaccagagacaccaccaccgcccgccattgcggacctgctcaaatcggtagacagtgaggctgtgagggagtactgcaaagaaaaaggatggttggttcctgagatcaactccaccctccaaattgagaaacatgtcagtcgggctttcatttgcttacaggcattgaccacatttgtgtcagtggctggaatcatatatataatatataagctctttgcgggttttcaaggtgcttatacaggagtgcccaaccagaagcccagagtgcctaccctgaggcaagcaaaagtgcaaggccctgcctttgagttcgccgtcgcaatgatgaaaaggaactcaagcacggtgaaaactgaatatggcgagtttaccatgctgggcatctatgacaggtgggccgttttgccacgccacgccaaacctgggccaaccatcttgatgaatgatcaagaggttggtgtgctagatgccaaggagctagtagacaaggacggcaccaacttagaactgacactactcaaattgaaccggaatgagaagttcagagacatcagaggcttcttagccaaggaggaagtggaggttaatgaggcagtgctagcaattaacaccagcaagtttcccaacatgtacattccagtaggacaggtcacagaatacggcttcctaaacctaggtggcacacccaccaagagaatgcttatgtacaacttccccacaagagcaggccagtgtggtggagtgctcatgtccaccggcaaggtactgggtatccatgttggtggaaatggccatcagggcttctcagcagcactcctcaaacactacttcaatgatgagcaaggtgaaatagaatttattgagagctcaaaggacgccgggtttccagtcatcaacacaccaagtaaaacaaagttggagcctagtgttttccaccaggtctttgaggggaacaaagaaccagcagtactcaggagtggggatccacgtctcaaggccaattttgaagaggctatattttccaagtatataggaaatgtcaacacacacgtggatgagtacatgctggaagcagtggaccactacgcaggccaactagccaccctagatatcagcactgaaccaatgaaactggaggacgcagtgtacggtaccgagggtcttgaggcgcttgatctaacaacgagtgccggttacccatatgttgcactgggtatcaagaagagggacatcctctctaagaagactaaggacctaacaaagttaaaggaatgtatggacaagtatggcctgaacctaccaatggtgacttatgtaaaagatgagctcaggtccatagagaaggtagcgaaaggaaagtctaggctgattgaggcgtccagtttgaatgattcagtggcgatgagacagacatttggtaatctgtacaaaactttccacctaaacccaggggttgtgactggtagtgctgttgggtgtgacccagacctcttttggagcaagataccagtgatgttagatggacatctcatagcatttgattactctgggtacgatgctagcttaagccctgtctggtttgcttgcctaaaaatgttacttgagaagcttggatacacgcacaaagagacaaactacattgactacttgtgcaactcccatcacctgtacagggataaacattactttgtgaggggtggcatgccctcgggatgttctggtaccagtattttcaactcaatgattaacaatatcataattaggacactaatgctaaaagtgtacaaagggattgacttggaccaattcaggatgatcgcatatggtgatgatgtgatcgcatcgtacccatggcctatagatgcatctttactcgctgaagctggtaagggttacgggctgatcatgacaccagcagataagggagagtgctttaacgaagttacctggaccaacgccactttcctaaagaggtattttagagcagatgaacagtaccccttcctggtgcatcctgttatgcccatgaaagacatacacgaatcaattagatggaccaaggatccaaagaacacccaagatcacgtgcgctcactgtgtctattagcttggcataacggggagcacgaatatgaggagttcatccgtaaaattagaagcgtcccagtcggacgttgtttgaccctccccgcgttttcaactctacgcaggaagtggttggactccttttagattagagacaatttgaaataatttagattggcttaaccctactgtgctaaccgaaccagataacggtacagtaggggtaaattctccgcattcggtgcgg

SEQ ID NO: 8 - DNA, 2537 bp, Homo sapiens
cgccgccgcgagccagtcgggagcgcgcgaggcgcggggagcctgggaccaggagcgagagccgcctacctgcagccgccgcccacggcacggcagccaccatggcgctcctgctgtgcttcgtgctcctgtgcggagtagtggatttcgccagaagtttgagtatcactactcctgaagagatgattgaaaaagccaaaggggaaactgcctatctgccgtgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatagagcctccatatgtctcatctgtgctctccgtgttcctttcctttttttgatatatgaaaacctattctggtctaaattgtgttactagcctcaaaatacatcaaaaaataagttaatcaggaactgtacggaatatatttttaaaaatttttgtttggttatatcgaaatagttacaggcactaaagttagtaaagaaaagtttaccatctgaaaaagctggattttctttaagaggttgattataaagttttctaaatttatcagtacctaagtaagatgtagcgctttgaatatgaaatcataggtgaagacatgggtgaacttacttgcataccaagttgatacttgaataaccatctgaaagtggtacttgatcatttttaccattatttttaggatgtgtatttcatttatttatggcccaccagtctcccccaaattagtacagaaatatccatgacaaaattacttacgtatgtttgtacttggttttacagctcctttgaaaactctgtgtttggaatatctctaaaaacatagaaaacactacagtggtttagaaattactaattttacttctaagtcattcataaaccttgtctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgctttatgttgttgttgtttttggatggtgttacatattatatgttctagaaacatgtaatcctaaatttaccctcttgaatataatccctggatgatattttttatcataaatgcagaataatcaaatacattttaagcaagttaagtgtcctccatcaattctgtattccagacttgggaggatgtacagttgctgttgtgtgatcaaacatgtctctgtgtagttccagcaaatcaagctgagctttgaaaaagtttgtcttagttttgtgaaggtgatttattcttaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - protein, 11 aa, synthetic sequence
ACDCRGDCFCG

SEQ ID NO: 10 - DNA, 1181 bp, Homo sapiens
ggcacgagggcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaaaaaaaaaaaaaaaaaaaaaaaaaaaaaaaaaaaaaaaaaa

SEQ ID NO: 11 - DNA, 2557 bp, Homo sapiens
gaattccgggagaagtgaccagagcaatttctgcttttcacagggcgggtttctcaacggtgacttgtgggcagtgccttctgctgagcgagtcatggcccgaaggcagaactaactgtgcctgcagtcttcactctcaggatgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccagctgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccatgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctagcaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcagaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttagacagcaaggccagatgcacatccctggaaggacatccatgttccgagaagaacagataatccctgtatttcaagacctctgtgcacttatttatgaacctgccctgctcccacagaacacagcaattcctcaggctaagctgccggttcttaaatccatcctgctaagttaatgttgggtagaaagagatacagagggg

SEQ ID NO: 12 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 13 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 14 - DNA, 1480 bp, Homo sapiens
gcggagaaagccagtgggaacccagacccataggagacccgcgtccccgctcggcctggccaggccccgcgctatggagttcctctgggcccctctcttgggtctgtgctgcagtctggccgctgctgatcgccacaccgtcttctggaacagttcaaatcccaagttccggaatgaggactacaccatacatgtgcagctgaatgactacgtggacatcatctgtccgcactatgaagatcactctgtggcagacgctgccatggagcagtacatactgtacctggtggagcatgaggagtaccagctgtgccagccccagtccaaggaccaagtccgctggcagtgcaaccggcccagtgccaagcatggcccggagaagctgtctgagaagttccagcgcttcacacctttcaccctgggcaaggagttcaaagaaggacacagctactactacatctccaaacccatccaccagcatgaagaccgctgcttgaggttgaaggtgactgtcagtggcaaaatcactcacagtcctcaggcccatgtcaatccacaggagaagagacttgcagcagatgacccagaggtgcgggttctacatagcatcggtcacagtgctgccccacgcctcttcccacttgcctggactgtgctgctccttccacttctgctgctgcaaaccccgtgaaggtgtatgccacacctggccttaaagagggacaggctgaagagagggacaggcactccaaacctgtcttggggccactttcagagcccccagccctgggaaccactcccaccacaggcataagctatcacctagcagcctcaaaacgggtcagtattaaggttttcaaccggaaggaggccaaccagcccgacagtgccatccccaccttcacctcggagggacggagaaagaagtggagacagtcctttcccaccattcctgcctttaagccaaagaaacaagctgtgcaggcatggtcccttaaggcacagtgggagctgagctggaaggggccacgtggatgggcaaagcttgtcaaagatgccccctccaggagagagccaggatgcccagatgaactgactgaaggaaaagcaagaaacagtttcttgcttggaagccaggtacaggagaggcagcatgcttgggctgacccagcatctcccagcaagacctcatctgtggagctgccacagagaagtttgtagccaggtactgcattctctcccatcctggggcagcactccccagagctgtgccagcaggggggctgtgccaacctgttcttagagtgtagctgtaagggcagtgcccatgtgtacattctgcctagagtgtagcctaaagggcagggcccacgtgtatagtatctgtatataagttgctgtgtgtctgtcctgatttctacaactggagtttttttatacaatgttctttgtctcaaaataaagcaatgtgttttttcgg

SEQ ID NO: 15 - DNA, 2323 bp, Homo sapiens
ctccctttgggcaaggacctgagacccttgtgctaagtcaagaggctcaatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctctcactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcactctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaaatcgcccttggtgaaagaaaattcttggaatactaaaaatcatgagatcctttaaatccttccatgaaacgttttgtgtggtggcacctcctacgtcaaacatgaagtgtgtttccttcagtgcatctgggaagatttctacctgaccaacagttccttcagcttccatttcgcccctcatttatccctcaacccccagcccacaggtgtttatacagctcagctttttgtcttttctgaggagaaacaaataagaccataaagggaaaggattcatgtggaatataaagatggctgactttgctctttcttgactcttgttttcagtttcaattcagtgctgtacttgatgacagacacttctaaatgaagtgcaaatttgatacatatgtgaatatggactcagttttcttgcagatcaaatttcacgtcgtcttctgtatactgtggaggtacactcttatagaaagttcaaaaagtctacgctctcctttctttctaactccagtgaagtaatggggtcctgctcaagttgaaagagtcctatttgcactgtagcctcgccgtctgtgaattggaccatcctatttaactggcttcagcctccccaccttcttcagccacctctctttttcagttggctgacttccacacctagcatctcatgagtgccaagcaaaaggagagaagagagaaatagcctgcgctgttttttagtttgggggttttgctgtttccttttatgagacccattcctatttcttatagtcaatgtttcttttatcacgatattattagtaagaaaacatcactgaaatgctagctgcaagtgacatctctttgatgtcatatggaagagttaaaacaggtggagaaattccttgattcacaatgaaatgctctcctttcccctgcccccagaccttttatccgacttacctagattctacatattctttaaatttcatctcaggcctccctcaaccccaccacttcttttataactagtcctttactaatccaacccatgatgagctcctcttcctggcttcttactgaaaggttaccctgtaacatgcaattttgcatttgaataaagcctgctttttaagtgtt

SEQ ID NO: 16 - DNA, 2615 bp, Homo sapiens
ccttttttggcctcgacggcggcaacccagcctccctcctaacgccctccgcctttgggaccaaccaggggagctcaagttagtagcagccaaggagaggcgctgccttgccaagactaaaaagggaggggagaagagaggaaaaaagcaagaatcccccacccctctcccgggcggagggggcgggaagagcgcgtcctggccaagccgagtagtgtcttccactcggtgcgtctctctaggagccgcgcgggaaggatgctggtccgcaggggcgcgcgcgagggcccaggatgccgcggggctggaccgcgctttgcttgctgagtttgctgccttctgggttcatgagtcttgacaacaacggtactgctaccccagagttacctacccagggaacattttcaaatgtttctacaaatgtatcctaccaagaaactacaacacctagtacccttggaagtaccagcctgcaccctgtgtctcaacatggcaatgaggccacaacaaacatcacagaaacgacagtcaaattcacatctacctctgtgataacctcagtttatggaaacacaaactcttctgtccagtcacagacctctgtaatcagcacagtgttcaccaccccagccaacgtttcaactccagagacaaccttgaagcctagcctgtcacctggaaatgtttcagacctttcaaccactagcactagccttgcaacatctcccactaaaccctatacatcatcttctcctatcctaagtgacatcaaggcagaaatcaaatgttcaggcatcagagaagtgaaattgactcagggcatctgcctggagcaaaataagacctccagctgtgcggagtttaagaaggacaggggagagggcctggcccgagtgctgtgtggggaggagcaggctgatgctgatgctggggcccaggtatgctccctgctccttgcccagtctgaggtgaggcctcagtgtctactgctggtcttggccaacagaacagaaatttccagcaaactccaacttatgaaaaagcaccaatctgacctgaaaaagctggggatcctagatttcactgagcaagatgttgcaagccaccagagctattcccaaaagaccctgattgcactggtcacctcgggagccctgctggctgtcttgggcatcactggctatttcctgatgaatcgccgcagctggagccccacaggagaaaggctgggcgaagacccttattacacggaaaacggtggaggccagggctatagctcaggacctgggacctcccctgaggctcagggaaaggccagtgtgaaccgaggggctcagaaaaacgggaccggccaggccacctccagaaacggccattcagcaagacaacacgtggtggctgataccgaattgtgactcggctaggtggggcaaggctgggcagtgtccgagagagcacccctctctgcatctgaccacgtgctacccccatgctggaggtgacatctcttacgcccaacccttccccactgcacacacctcagaggctgttcttggggccctacaccttgaggagggggcaggtaaactcctgtcctttacacattcggctccctggagccagactctggtcttctttgggtaaacgtgtgacgggggaaagccaaggtctggagaagctcccaggaacaatcgatggccttgcagcactcacacaggacccccttcccctaccccctcctctctgccgcaatacaggaacccccaggggaaagatgagcttttctaggctacaattttctcccaggaagctttgatttttaccgtttcttccctgtattttctttctctactttgaggaaaccaaagtaaccttttgcacctgctctcttgtaatgatatagccagaaaaacgtgttgccttgaaccacttccctcatctctcctccaagacactgtggacttggtcaccagctcctcccttgttctctaagttccactgagctccatgtgccccctctaccatttgcagagtcctgcacagttttctggctggagcctagaacaggcctcccaagttttaggacaaacagctcagttctagtctctctggggccacacagaaactctttttgggctcctttttctccctctggatcaaagtaggcaggaccatgggaccaggtcttggagctgagcctctcacctgtactcttccgaaaaatcctcttcctctgaggctggatcctagccttatcctctgatctccatggcttcctcctccctcctgccgactcctgggttgagctgttgcctcagtcccccaacagatgcttttctgtctctgcctccctcaccctgagccccttccttgctctgcacccccatatggtcatagcccagatcagctcctaacccttatcaccagctgcctcttctgtgggtgacccaggtccttgtttgctgttgatttctttccagaggggttgagcagggatcctggtttcaatgacggttggaaatagaaatttccagagaagagagtattgggtagatattttttctgaatacaaagtgatgtgtttaaatactgcaattaaagtgatactgaaacac

SEQ ID NO: 17 - DNA, 5133 bp, Homo sapiens
cctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaatggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctctacccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgaatgtctcctttgcaggctcatagggaagacctgctgagggaccagccaagagggctgcaaaagtgagggcttgtcattaccagacggttcaccagcctctcttggttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgcccacccctccttccttgaccagcagatcccagctcacgtcacacacttggttgggtcctcacatctttcacacttccaccaccctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgacgccgcatgatatgtcagggcgtgggacatctagtaggtgcttgacataatttcactgaattaatgacagagccagtgggaagatacagaaaaagagggccggggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccggacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaa

SEQ ID NO: 18 - DNA, 4740 bp, Homo sapiens
gaattccgtggttcctcagtggtgcctgcaacccctggttcacctccttccaggttctggctccttccagccatggctctcagagtccttctgttaacagccttgaccttatgtcatgggttcaacttggacactgaaaacgcaatgaccttccaagagaacgcaaggggcttcgggcagagcgtggtccagcttcagggatccagggtggtggttggagccccccaggagatagtggctgccaaccaaaggggcagcctctaccagtgcgactacagcacaggctcatgcgagcccatccgcctgcaggtccccgtggaggccgtgaacatgtccctgggcctgtccctggcagccaccaccagcccccctcagctgctggcctgtggtcccaccgtgcaccagacttgcagtgagaacacgtatgtgaaagggctctgcttcctgtttggatccaacctacggcagcagccccagaagttcccagaggccctccgagggtgtcctcaagaggatagtgacattgccttcttgattgatggctctggtagcatcatcccacatgactttcggcggatgaaggagtttgtctcaactgtgatggagcaattaaaaaagtccaaaaccttgttctctttgatgcagtactctgaagaattccggattcactttaccttcaaagagttccagaacaaccctaacccaagatcactggtgaagccaataacgcagctgcttgggcggacacacacggccacgggcatccgcaaagtggtacgagagctgtttaacatcaccaacggagcccgaaagaatgcctttaagatcctagttgtcatcacggatggagaaaagtttggcgatcccttgggatatgaggatgtcatccctgaggcagacagagagggagtcattcgctacgtcattggggtgggagatgccttccgcagtgagaaatcccgccaagagcttaataccatcgcatccaagccgcctcgtgatcacgtgttccaggtgaataactttgaggctctgaagaccattcagaaccagcttcgggagaagatctttgcgatcgagggtactcagacaggaagtagcagctcctttgagcatgagatgtctcaggaaggcttcagcgctgccatcacctctaatggccccttgctgagcactgtggggagctatgactgggctggtggagtctttctatatacatcaaaggagaaaagcaccttcatcaacatgaccagagtggattcagacatgaatgatgcttacttgggttatgctgccgccatcatcttacggaaccgggtgcaaagcctggttctgggggcacctcgatatcagcacatcggcctggtagcgatgttcaggcagaacactggcatgtgggagtccaacgctaatgtcaagggcacccagatcggcgcctacttcggggcctccctctgctccgtggacgtggacagcaacggcagcaccgacctggtcctcatcggggccccccattactacgagcagacccgagggggccaggtgtccgtgtgccccttgcccagggggcagagggctcggtggcagtgtgatgctgttctctacggggagcagggccaaccctggggccgctttggggcagccctaacagtgctgggggacgtaaatggggacaagctgacggacgtggccattggggccccaggagaggaggacaaccggggtgctgtttacctgtttcacggaacctcaggatctggcatcagcccctcccatagccagcggatagcaggctccaagctctctcccaggctccagtattttggtcagtcactgagtgggggccaggacctcacaatggatggactggtagacctgactgtaggagcccaggggcacgtgctgctgctcaggtcccagccagtactgagagtcaaggcaatcatggagttcaatcccagggaagtggcaaggaatgtatttgagtgtaatgatcaggtggtgaaaggcaaggaagccggagaggtcagagtctgcctccatgtccagaagagcacacgggatcggctaagagaaggacagatccagagtgttgtgacttatgacctggctctggactccggccgcccacattcccgcgccgtcttcaatgagacaaagaacagcacacgcagacagacacaggtcttggggctgacccagacttgtgagaccctgaaactacagttgccgaattgcatcgaggacccagtgagccccattgtgctgcgcctgaacttctctctggtgggaacgccattgtctgctttcgggaacctccggccagtgctggcggaggatgctcagagactcttcacagccttgtttccctttgagaagaattgtggcaatgacaacatctgccaggatgacctcagcatcaccttcagtttcatgagcctggactgcctcgtggtgggtgggccccgggagttcaacgtgacagtgactgtgagaaatgatggtgaggactcctacaggacacaggtcaccttcttcttcccgcttgacctgtcctaccggaaggtgtccacactccagaaccagcgctcacagcgatcctggcgcctggcctgtgagtctgcctcctccaccgaagtgtctggggccttgaagagcaccagctgcagcataaaccaccccatcttcccggaaaactcagaggtcacctttaatatcacgtttgatgtagactctaaggcttcccttggaaacaaactgctcctcaaggccaatgtgaccagtgagaacaacatgcccagaaccaacaaaaccgaattccaactggagctgccggtgaaatatgctgtctacatggtggtcaccagccatggggtctccactaaatatctcaacttcacggcctcagagaataccagtcgggtcatgcagcatcaatatcaggtcagcaacctggggcagaggagcctccccatcagcctggtgttcttggtgcccgtccggctgaaccagactgtcatatgggaccgcccccaggtcaccttctccgagaacctctcgagtacgtgccacaccaaggagcgcttgccctctcactccgactttctggctgagcttcggaaggcccccgtggtgaactgctccatcgctgtctgccagagaatccagtgtgacatcccgttctttggcatccaggaagaattcaatgctaccctcaaaggcaacctctcgtttgactggtacatcaagacctcgcataaccacctcctgatcgtgagcacagctgagatcttgtttaacgattccgtgttcaccctgctgccgggacagggggcgtttgtgaggtcccagacggagaccaaagtggagccgttcgaggtccccaaccccctgccgctcatcgtgggcagctctgtcgggggactgctgctcctggccctcatcaccgccgcgctgtacaagctcggcttcttcaagcggcaatacaaggacatgatgagtgaagggggtcccccgggggccgaaccccagtagcggctccttcccgacagagctgcctctcggtggccagcaggactctgcccagaccacacgtagcccccaggctgctggacacgtcggacagcgaagtatccccgacaggacgggcttgggcttccatttgtgtgtgtgcaagtgtgtatgtgcgtgtgtgcgagtgtgtgcaagtgtctgtgtgcaagtgtgtgcacgtgtgcgtgtgcgtgcatgtgcactcgcacgcccatgtgtgagtgtgtgcaagtatgtgagtgtgtccagtgtgtgtgcgtgtgtccatgtgtgtgcagtgtgtgcatgtgtgcgagtgtgtgcatgtgtgtgctcaggggctgtggctcacgtgtgtgactcagagtgtctctggcgtgtgggtaggtgacggcagcgtagcctctccggcagaagggaactgcctgggctcccttgtgcgtgggtaagccgctgctgggttttcctccgggagaggggacggtcaatcctgtgggtgaagagagagggaaacacagcagcatctctccactgaaagaagtgggacttcccgtcgcctgcgagcctgcggcctgctggagcctgcgcagcttggatggatactccatgagaaaagccgtgggtggaaccaggagcctcctccacaccagcgctgatgcccaataaagatgcccactgaggaatcatgaagcttcctttctggattcatttattatttcaatgtgactttaattttttggatggataagcctgtctatggtacaaaaatcacaaggcattcaagtgtacagtgaaaagtctccctttccagatattcaagtcacctccttaaaggtagtcaagattgtgttttgaggtttccttcagacagattccaggcgatgtgcaagtgtatgcacgtgtgcacacaccacacacatacacacacacaagcttttttacacaaatggtagcatactttatattggtctgtatcttgctttttttcaccaatatttctcagacatcggttcatattaagacataaattactttttcattcttttataccgctgcatagtattccattgtgtgagtgtaccataatgtatttaaccagtcttcttttgatatactattttcatctcttgttattgcatctgctgagttaataaatcaaatatatgtcaaaaaaaaaaaaaaaaaaaaaaaaaaaaaaaaaaaaaaaaa